(19) United States
(12) Patent Application Publication
(10) Pub. No.: US 2024/0068058 A1
(43) Pub. Date: Feb. 29, 2024

(54) HPV Detection from urine analyte in male, non-binary and transgender patients

(71) Applicant: Brian Joseph Haijar, Palisades Park, NJ (US)

(72) Inventor: Brian Joseph Haijar, Palisades Park, NJ (US)

(21) Appl. No.: 18/445,222

(22) Filed: Jun. 2, 2023

Related U.S. Application Data
(60) Provisional application No. 63/474,639, filed on Aug. 31, 2022.

(51) Int. Cl. C12Q 1/70; C12Q 1/6844

(52) U.S. Cl. CPC C12Q 1/708; C12Q 1/6844

(57) ABSTRACT

The present invention allows for diagnosis of HPV in urine analyte in male, non-binary and transgender patients using a PCR-based assay. The Cobas HPV test can detect one or more high-risk forms of HPV 16, 18, 68 and other subtypes in the Cobas® 6800/8800 HPV assay using urine analyte in male, non-binary, and transgender patients with high analytical sensitivity, specificity and reproducibility. The method is useful for mass screening and for specimen self-sampling via a diagnostic kit.

### CROSS REFERENCE TO RELATED APPLICATIONS

This application claims the benefit of priority to U.S. Provisional Application Ser. No. 63/474,639, filed Aug. 31, 2022, the contents of which are incorporated by reference herein in their entirety.

### TECHNICAL FIELD

The present invention is directed to clinical diagnostics based on molecular techniques.

### BACKGROUND

Human Papillomavirus (HPV), a member of the Papillomaviridae, is a non-enveloped double stranded DNA virus which is classified into low-risk HPVs involved in causing anogenital and cutaneous warts, and high risk HPVs (16, 18, 31, 33, 39, 45, 51, 52, 56, 58, 59, 66, 68) that are oncogenic and their persistent infections are responsible for oropharyngeal and anogenital cancers. HPV is the most common sexually transmitted virus. According to the Centers for Disease Control and Prevention (CDC), 79 million American men and women are infected with HPV. HPV is also highly prevalent in male patients. Although considerable attention has been provided to gynecologic HPV disease, men can be affected by HPV-related cancers of the anus, penis and oropharynx; genital warts; common warts on the hands, fingers or face; and plantar warts. Every year over 12,000 men are affected by cancers caused by HPV. Forty different types of HPV have been identified in anogenital infections. Nearly 4 out of every 10 cases of cancer caused by HPV occur in men.

Most HPV infections are asymptomatic. Since most HPV infections do not show any symptoms, routine screening is essential for the prevention and treatment of sexually transmitted infections. HPV-based screening is more effective than cytology screening in preventing cervical cancer in large randomized controlled trials.

According to Mayo clinic there is no male PCR test for HPV available in USA. The HPV test is available only to women using clinician-obtained cervical samples. One test uses the Cobas 6800, an automated PCR-based sensitive HPV detection platform using clinician-obtained cervical samples. The test uses DNA amplification and hybridization to detect 14 high-risk HPV genotypes, including HPV 16 and HPV 18. The sampling process for HPV detection is often found to be uncomfortable, and women, especially female children and young girls, feel reluctant and embarrassed to go through Pap smears and vaginal examinations. No currently validated method to test urine analyte for HPV in male patients exists. The Cobas HPV test specifies that it is for women since the only analyte used for testing is Thinprep and pap smear which comes from the female cervical area. A gender neutral HPV test, with no age limitations, which is urine-based instead of traditional Thinprep/pap smear is needed.

Anther limitation of the Cobas HPV testing is that it is gender specific for women. HPV screening is equally important for males and non-binary people, especially those with an intact cervix as well as transgender people. Additionally, no HPV test exists to detect the virus in men, non-binary people and transgender people. However, men, non-binary people and transgender people can be infected with HPV and pass the virus to their sex partners. Moreover, the female focused-screening misses the fact that both cisgender and transgender men act as carriers for the transmission or HPV to women and other men through sexual intercourse.

Currently available HPV tests may lead to inconsistent results with men. The test requires collection of exfoliated cells from the external genitalia and it is difficult to obtain an adequate cell sample to test from the thick skin on the penis. Additionally, the test is variable based on the sampling method used to collect the cells and the sensitivity of the assay used for detection of HPV DNA. As a result, male patients are typically screened clinically with a visual inspection to check for lesions such as warts. Alternatives are peniscopy and urethroscopy which are invasive procedures. Men can obtain an anal swab for HPV but the CDC does not recommend routine testing for HPV in men.

There is a need for development of a robust PCR test via urine to detect HPV in male patients, transgender men and women and other nonbinary people before they can develop complicated disease or transmit this viral pathogen to their partners during sexual intercourse. Previous, PCR tests are gender-biased since they can only be applied for female patients. There is a need for a gender-neutral test for HPV.

In women, nucleic acid (DNA) testing by PCR is a non-invasive method for determining the presence of a cervical HPV infection. In men, PCR-based testing of tissue obtained from biopsy from penile epithelium and urethral cytobrush samples and other invasive techniques for sampling exfoliated cells have poor reliability. Problems associated with these tests include insufficient collection of cells for detection of HPV DNA by use of swabs and brushes. Additionally, urine samples in men have poor specimen adequacy and lack of HPV positivity. Even in female patients, urinary HPV detection is inaccurate. The problem has been the presence of soluble PCR inhibitors in urine. PCR urine assays for HPV are insufficient to efficiently and accurately identify HPV in male patients. The epidemiology of HPV infection in men is an important issue for men's health.

### SUMMARY

A first aspect relates to a method for determining the presence or absence of HPV nucleic acid molecules using one or more polymerase chain-reaction (PCR) molecular amplification tests in urine samples from male patients, non-binary patients and transgender patients.

The method comprises:
providing a unaltered urine specimen from a male patient;
extracting DNA from a cell;
using a PCR amplification process;
testing the DNA by Cobas® HPV test using Cobas® 6800 or 8800 assay; and
determining a positive or negative result for HPV detection.

The method comprises:
providing a unaltered urine specimen from a non-binary patient;
extracting DNA from a cell;
using a PCR amplification process;
testing the DNA by Cobas® HPV test using Cobas® 6800 or 8800 assay; and
determining a positive or negative result for HPV detection.

The method comprises:
providing a unaltered urine specimen from a transgender patient;
extracting DNA from a cell;
using a PCR amplification process;
testing the DNA by Cobas® HPV test using Cobas® 6800 or 8800 assay; and
determining a positive or negative result for HPV detection.

A first aspect also relates to a PCR-based diagnostic test for HPV from urine samples in male patients, transgender males and non-binary patients.

Optionally, in any of the preceding aspects, there is a PCR-based method which can detect one or more high-risk forms of HPV 16, 18, 68 and other subtypes in the Cobas® 6800/8800 HPV assay in male patients with high analytical sensitivity.

In another aspect, there is a gender-neutral PCR-based method which can detect one or more high-risk forms of HPV 16, 18, 68 and other subtypes in the Cobas® 6800/8800 HPV assay in children (e.g. victims of abuse), adolescents, non-binary people, and transgender people with high analytical sensitivity.

In one aspect, the Cobas 6800 detects both HPV 16 and HPV 18 at a minimum concentration of 50 copies/mL with an average Ct value of 37 and 35.6, respectively.

In another aspect, the method provides clinical validation for the Cobas 6800/8800 HPV test for male patients.

In yet another aspect, a semi-quantitative test for HPV viral load in urine is provided which is highly specific and reproducible detection for detection of HPV in asymptomatic men.

In yet another aspect, the method is able to automatically perform the identification of HPV in men.

In yet another aspect, the test is a non-invasive, easily accessible, and useful screening test capable of detecting subclinical HPV infection in men easy for clinicians to perform without great cost or discomfort to patients. The test can be performed more frequently than current screening methods.

In another aspect, the method is an in vitro detection of HPV in clinician-collected urine specimens.

In another aspect, the method provides HPV testing in men useful for identifying female partners who are at increased risk for development of cervical cancer. This, in turn, will provide the opportunity to develop pathways for the management of positive test results including instituting appropriate medical therapy and reducing health care costs.

In another aspect, the method provides a non-invasive home-based urine collection PCR-based method which allows for men, non-binary and transgender patients to self-sample and return the sample to a laboratory for HPV testing by mail.

Optionally, in one aspect, a report is generated and sent to a physician/patient for review.

In another aspect, the method provides male patients, non-binary patients, children, or transgender patients with a most preferred HPV self-sampling urine collection screening option.

In another aspect the method provides PCR identification of HPV which correlates with clinical significance.

In another aspect, the method is comparable in sensitivity, specificity, accuracy, and overall percent agreement to ThinPrep and other cervical testing methods.

In another embodiment, the PCR technology platform is integrated with data management systems, locally, regionally and nationally to allow for effective epidemiological surveillance, mass screening, and control of disease outbreaks.

In some embodiments, the patient will be unvaccinated, since vaccinated patients have reduced prevalence of HPV 16 and 18.

In another embodiment, the patient may be seen in an acute care setting such as an emergency department or hospital.

In another aspect, the method comprises a diagnostic kit for HPV detection, PCR sequencing and analysis. In some embodiments, the kit incudes instructions relating to its use. In some embodiments, the method is a home-based test.

In one embodiment of the present invention, a kit is used for diagnosing or confirming with connectivity through the Cloud, an App, a computer, artificial intelligence (AI) or through other electronic means.

In some embodiments, nucleic acid amplification and detection methods useful for the diagnosis and management of a variety of HPV strains considered to be high-risks can be used.

This Summary is provided to introduce a selection of concepts in a simplified form that are further described below in the Detailed Description. This Summary is not intended to identify key features or essential features of the claimed subject matter, nor is it intended to be used as an aid in determining the scope of the claimed subject matter. The claimed subject matter is not limited to implementations that solve any or all disadvantages noted in the Background.

### BRIEF DESCRIPTION OF THE DRAWINGS

FIG. 1 shows the HPV urine clinical validation summary. Based on Limit of Detection (LOD) data low copy number of 25 copies/ml, mid 500 copies/ml and high 1000 copies/ml of the determined LOD and 10 negative samples.

FIG. 2 illustrates the stability study for determining the stability of urine samples at room temperature 20-25° C.

FIG. 3 illustrates the Semi-Quantitative test for viral load categories according to example embodiments of the present disclosure.

FIG. 4 presents LOD data for HPV 16, HPV 18 and HPV 68.

FIG. 5 presents a pilot validation study on 173 clinical urine samples for HPV detection.

FIG. 6 presents the data on accuracy and reproducibility of urine-based HPV detection using the Cobas.

FIG. 7 presents the dilution series used to establish the LOD for HPV in urine samples.

FIG. 8 Presents the data comparison of the ThinPrep Cervical Test to the Urine-based HPV detection using Cobas 6800.

### DETAILED DESCRIPTION

The disclosure provides methods for the preparation and detection of HPV in urine samples provided by male patients.

The disclosure also provides methods for the preparation and detection of HPV in children and adolescents, non-binary people, men who have sex with men (cisgender MSM), and transgender individuals. HPV prevalence is high among MSM and several studies have reported even higher prevalence in transgender women. Vaccination has been recommended by the Advisory Committee on Immunization Practices for males since 2010, for transgender people since 2016 and for children (can start at 9 years old) and adolescents at age 11 or 12 years. Although vaccination is the primary prevention for HPV, vaccination rates among the lesbian, gay, transgender, and bisexual (LGBT) populations remain low. Secondary prevention for HPV includes Pap tests and HPV testing. Pap smears on transgender men have a ten-fold higher incidence of an unsatisfactory result compared to non-transgender women.

The terms patient, people, person and individuals are used interchangeably throughout.

The term nonbinary is defined as relating to or being a person who identifies with or expresses a gender identity that is neither entirely male nor entirely female.

The term transgender is defined as a person whose gender identity differs from the sex the person had or was identified as having at birth especially, relating to, or being a person whose gender identity is opposite the sex the person had or was identified as having at birth.

The term cisgender is defined as a person whose gender identity corresponds with the sex the person had or was identified as having at birth.

In one aspect of the disclosure, the gender bias of current HPV testing methods, which predominantly focus on individuals with female anatomy are addressed. Presently, HPV molecular detection is primarily designed for females, leading to low screening rates among men and non-binary individuals. Recognizing the significance of HPV screening for individuals on the female-to-male, male-to-female spectrum, and other genders, it becomes crucial to provide inclusive screening options. Additionally, cisgender males, particularly those who identify as homosexual, are prone to chronic HPV infections and can serve as carriers, transmitting the virus to females and other males through sexual contact. Hence, broadening the scope of HPV screening to encompass these populations becomes imperative. Another drawback of the current method lies in the invasive specimen collection process, causing discomfort and exacerbating genital dysphoria. In one aspect of the present disclosure, the PCR-based urine assay for HPV is useful for detection in these patient populations.

In one aspect of the disclosure, is provided a non-invasive alternative that ensures a more comfortable screening experience for all individuals regardless of gender. The method is gender-neutral solution and addresses these limitations and offers a comprehensive and inclusive approach to HPV screening. In one aspect of the present disclosure, the PCR-based urine assay for HPV is useful for detection in these patient populations.

LGBT populations face a unique set of health challenges, including barriers to achieving optimal preventive/screening for HPV and lack of access to health care. Individuals who have never or have rarely been screened for cervical cancer are at the highest risk for progression of chronic HPV infection to malignancy, morbidity and mortality. In one aspect of the present disclosure, the PCR-based urine assay for HPV is useful for detection in these patient populations.

Aspects of the disclosure described herein may aid HPV testing methods in men and lead to improved patient outcomes. The availability of a HPV urine test in as few hours or less, as opposed to a few days, could potentially decrease transmissibility.

Children as young as 9 years old and/or victims of sexual assault and adolescents are among the groups for whom the HPV vaccine is routinely recommended. Uptake of the vaccine remains low in these patient populations. In one aspect of the present invention, the PCR-based urine assay for HPV is useful for detection in these patient populations.

Aspects of the disclosure described herein overcome disadvantages associated with current strategies and provides tools, and other methods to facilitate and simplify PCR urine testing for HPV in male, transgender and nonbinary patients.

Certain embodiments of the present invention include, but are not limited to, the method of the invention as follows:
1) Providing an unaltered urine specimen from a male patient
2) DNA is prepared and extracted from cell (extraction process)
3) PCR amplification process (Cycle Thresholds)
4) DNA tested by Cobas® HPV test using Cobas® 6800 or 8800 assay
5) Sample determination as positive or negative for HPV detection was calculated
6) Report is generated and send to physician/patient for review

Certain embodiments of the present invention include, but are not limited to, the method of the invention as follows:
1) Providing an unaltered urine specimen from a child or adolescent, or a non-binary transgender male patient.
2) DNA is prepared and extracted from cell (extraction process)
3) PCR amplification process (Cycle Thresholds)
4) DNA tested by Cobas® HPV test using Cobas® 6800 or 8800 assay
5) Sample determination as positive or negative for HPV detection was calculated
6) Report is generated and send to physician/patient for review

The specimen in step one is preferably a first void urine sample. However, the test is valid without the need for first void urines. Use of first void urine does reduce rates of invalid calls. The urine sample is collected via Specimen Container McKesson 120 mL. The sample is stored at 2-30° C. for up to 7 days prior to performing the Cobas® HPV test. The sample is not preserved or centrifuged into a pellet. 1 mL of the sample is loaded on Cobas for amplification.

The main reason for using Cobas 6800 was that it was approved by FDA for Thinprep and pap smear samples for HPV detection. It is understood that the assay may also be performed on the Cobas 8800 system. The assay may also be performed on future version of the Cobas series or improved PCR machines.

The success of screening for HPV depends on the reliability and sensitivity of the test used. The Cobas HPV test is based on a fully automated sample preparation (nucleic acid extraction and purification) followed by PCR amplification and detection. The Cobas HPV test for use on the 6800 system has not been validated using urine analyte. Cobas 6800 is FDA approved for cervical screening for HPV in women. The Cobas HPV test processed on the Cobas 6800 system has automated sample preparation and high-throughput (over 1,000 samples in 24 hours).

The Cobas® 6800/8800 Systems consists of the sample supply module, the transfer module, the processing module, and the analytic module. Automated data management is performed by the Cobas® 6800/8800 software which assigns test results for all tests as positive, negative, or invalid. Results can be reviewed directly on the system screen and exported or printed as a report. The assay has a total duration of 3 hours. The Cobas® 6800 assay is a PCR-based method, separately detecting HPV 16, HPV 18, and HPV 68 including the B-globin gene as an extraction and amplification control. HPV 16 and HPV 18 are the two most oncogenic genotypes of HPV.

Throughout this disclosure, the terms Cobas 6800 and 8800 are used interchangeably without limitation. Other improved assays may be employed.

The test has the ability to test for other serotypes including HPV 31, 33, 35, 39, 45, 52, 56, 58, 59, 66. PCR amplification and detection occur in a single tube, where probes with different reporter dyes track the different targets in the reaction with B-globin as the control for extraction and amplification adequacy.

The nucleic acid (DNA) from patient samples is extracted. The urine sample is treated to release the nucleic acid by addition of proteinase and lysis reagent The released nucleic acid binds to the silica surface of the added magnetic glass particles.

Unbound substances and impurities, such as denatured protein, cellular debris and potential PCR inhibitors are removed with subsequent wash steps.

Purified nucleic acid is eluted from the magnetic glass particles with elution buffer at elevated temperature. Other solid particles may be used.

External controls (positive and negative) are processed in the same way with each Cobas® HPV run. HPV positive ingredients include: Tris buffer, <0.05% EDTA, <0.1% Sodium azide, <0.01% Non-infectious plasmid DNA (microbial) containing HPV16, HPV18 and HPV39 sequences, <0.01% Non-infectious plasmid DNA (microbial) containing β-globin sequences, <0.002% Poly rA RNA (synthetic).

### Example 1 Accuracy for Negative Controls

The expected outcomes should be that all negative controls are all negative. Therefore, the accuracy should be at 100% for the negative controls.

Negative Control Samples
1. ENTC=ENTC Sample with transport medium only.
2. DNTC=NTC Sample with nuclease free water only.

### Example 2 Accuracy for Positive Controls

All known positive control samples should be positive to reach 100% accuracy. The acceptable range is 95-100%. The accuracy should be at 100% for the positive controls.

A thermostable DNA polymerase enzyme is used for PCR amplification. The HPV and β-globin sequences. The control is used to determine the quality of the sample DNA and the presence of potential inhibitory substances. The control indicates whether the DNA extraction was successful and whether the PCR works. For a sample to be valid this control gene needs to be amplified simultaneously utilizing a universal PCR amplification profile with predefined temperature steps and number of cycles.

The master mix includes deoxy uridine triphosphate (dUTP), instead of deoxythymidine triphosphate (dTTP), which is incorporated into the newly synthesized DNA (amplicon). Any contaminating amplicon from previous PCR runs are eliminated by the AmpErase® enzyme (uracil N-glycosylase (UNG), which is included in the PCR master mix, during the first thermal cycling step. However, newly formed amplicon are not eliminated since the AmpErase® enzyme is inactivated once exposed to temperatures above 55° C. Cobas® HPV master mix contains detection probes specific for twelve high-risk HPV target sequences. These are primers for detecting 12 HPV serotypes: one detection probe specific for the HPV 16 target sequence, one detection probe specific for the HPV 18 target sequence and one for β-globin. The amplified signal from high-risk HPV 68 type is detected using the same fluorescent dye while HPV 16, HPV 18 and β-globin signals are each detected with their own dedicated fluorescent dye. When not bound to the target sequence, the fluorescent signal of the intact probes is suppressed by a quencher dye. During the PCR amplification step, hybridization of the probes to the specific single-stranded DNA template results in cleavage of the probe by the 5′ to 3′ exonuclease activity of the DNA polymerase resulting in separation of the reporter and quencher dyes and the generation of a fluorescent signal. With each PCR cycle, increasing amounts of cleaved probes are generated and the cumulative signal of the reporter dye increases concomitantly.

Real-time detection and discrimination of PCR products (i.e. amplified DNA amplicon or resulting DNA sequence from PCR reaction) is accomplished by measuring the fluorescence of the released reporter dyes for the HPV targets and β-globin, respectively. The Cobas® 6800 System consists of the sample supply module, the transfer module, the processing module, and the analytic module. Automated data management is performed by the Cobas® 6800 software which assigns test results for all tests as positive, negative, or invalid. Positive and negative refer to the determination of the Cobas 6800 instrument.

In real-time PCR, the DNA is amplified and simultaneously quantified. Variations of the general PCR method are known in the art. Results can be reviewed directly on the system screen and exported or printed as a report.

Example embodiments will now be described more fully hereinafter with reference to the accompanying figures. That which is encompassed by the claims may, however, be embodied in many different forms and should not be construed as limited to the embodiments set forth herein; rather, these embodiments are provided by way of example.

Validation Strategy and Summary

A LOD pilot study is used to explore and confirm the LOD of the HPV testing. The limit of detection (LOD) of the assay was established by running dilutions (1.25-10,000 copies/ml). A dilution series of 10,000 copies/mL, 5000 copies/mL, 1000 copies/mL 500 copies/mL, 250 copies/mL 125 copies/mL, 50 copies/mL 25 copies/mL, 5 copies/mL, 2.5 copies/mL and 1.25 copies/mL using whole organism HPV 1, 18, and 68 along with internal positive and negative controls (Exact Diagnostics LLC), non-template, and negative extraction controls. All the samples were run on Cobas® 6800 three times. Three replicates of each concentration were processed over a period of three days (on three different days) to gauge the possible LOD of the HPV testing. The Ct values for assay replicates were averaged, and the values were plotted. Based on the Ct cutoff values of all three HPV types (16, 18 and 68) the limit of detection was established. The LOD was determined to be between 1.25 and 10,000 copies/mL, depending on the uniformity of Ct value of detectable copy number.

The Cobas 6800 is an automated high-throughput real-time PCR based system which is used for routine cervical cancer screening and detects 14 types of high-risk HPV (HPV-31, 33, 35, 39, 45, 51, 52, 58, 59, 66 and 68) along with HPV16 and HPV18. The assay starts with nucleic acid extraction and release from the samples which then binds to the silica surface of the added magnetic glass particles. The purified nucleic acid is then amplified by PCR reaction using thermostable DNA polymerase. The HPV and β-globulin sequences are amplified simultaneously a universal PCR amplification profile with predefined temperature steps and number of cycles. The automated management software assigns the results for the tests as positive, negative or invalid.

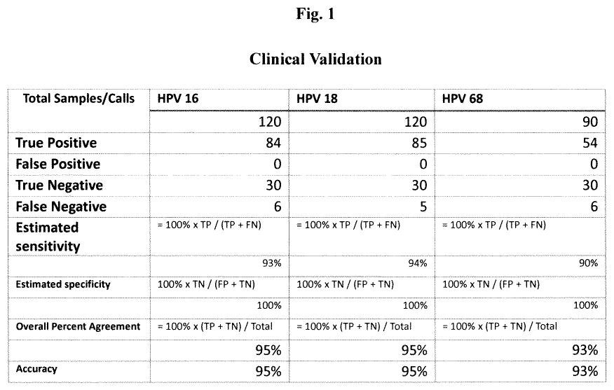

FIG. 1 shows the HPV urine clinical validation summary. Based on LOD data low copy number of 25 copies/ml, mid 500 copies/ml and high 1000 copies/ml of the determined LOD and 10 negative samples. The clinical performance of the Cobas 6800 on urine samples was evaluated by calculating the sensitivity, specificity, accuracy, and overall percent agreement. The validation study was performed over three days for each positive and negative sample to evaluate the clinical performance of the assay. For HPV 16, the sensitivity was calculated to be 93% with 100% specificity, as no false positive was recorded. Similarly, the sensitivity for HPV 18 was established to be 94% with 100% specificity. For HPV 68, the sensitivity was calculated to be 90% and the specificity was 100%, similar to that of HPV 16 and HPV 18. The overall percent agreement and accuracy for HPV 16 and HPV 18 were calculated to be 95%. However, both the overall percent agreement and accuracy were 93% for HPV 68.

For the clinical validation study as shown in FIG. 1, 40 samples (based on LOD data low copy number of 25 copies/ml, mid 500 copies/ml and high 10,000 copies/ml with a final volume of 1 mL) out of which 30 were spiked positive samples and 10 were negative control samples were tested on Cobas® 6800 to examine sensitivity, specificity, percent agreement and accuracy. The positive samples were spiked with different concentrations of the HPV whole organism controls (Exact Diagnostics LLC). The specimen type for evaluation was HPV 16, 18 and 68 positive spiked urine samples. For achieving highest levels of confidence there should be no contamination in the negative controls (ENTC and NTC) and the positive Controls should matched. Experiments were carried out over a period of 3 days by two different technicians. All known spiked positive and negative samples were run in triplicates over three days. Two out of three calls needed to be in concordance with one another to support a “detected” call for HPV. Each set of triplicates was evaluated to make a single positive or negative call for confidence.

The clinical validation study was performed over three days where each spiked sample was run daily to get three readings. A total of 40 positive spiked samples (based on LOD data low copy number of 25 copies/ml, mid 500 copies/ml and high 1000 copies/ml of the determined LOD) and 10 negative samples were tested on Cobas® 6800 to examine the overall performance of the assay for clinical samples. In order to validate this Cobas® 6800 test for HPV 16, 18 and 68, the following performance characteristics were evaluated:
1. Accuracy
2. Sensitivity
3. Specificity
4. Percent Agreement

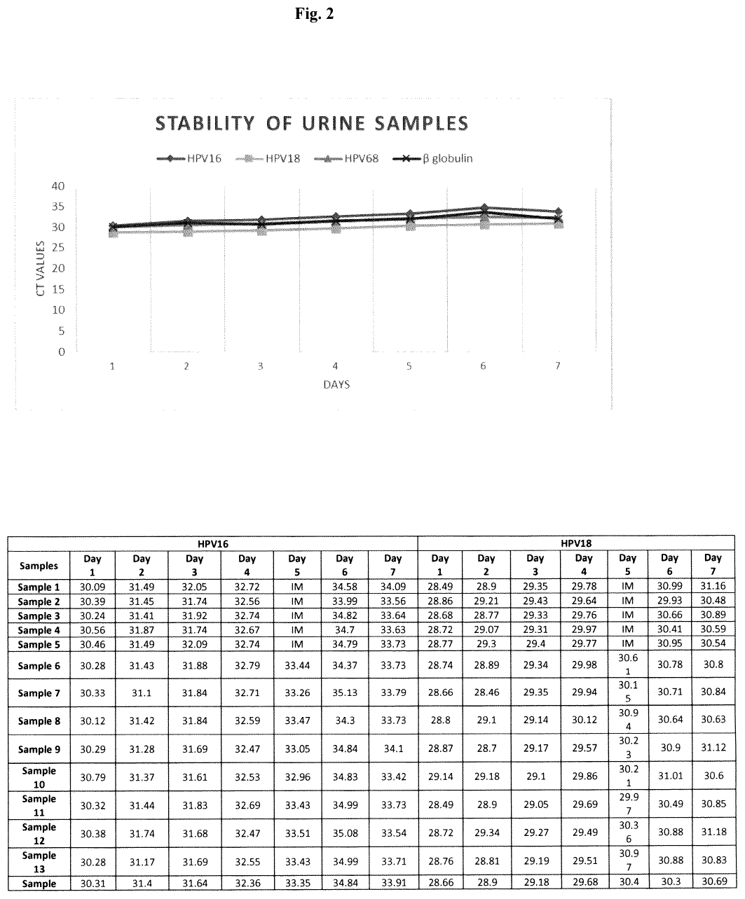

FIG. 2 illustrates the stability study for determining the stability of urine samples at room temperature 20-25° C. The stability of the assay using urine samples was analysed with a total of 20 samples, including 5 HPV-negative and 15 HPV-spiked positive samples over a course of seven days. The stability was measured based on the detection of β globulin, and the urine sample was considered valid as long as the β globulin was detectable according to the protocol provided by the manufacturer, Roche Diagnostics. The Ct values were recorded and plotted for the complete duration of the stability experiment. However, the day 5 stability data for samples 1-5 was lost due to instrument malfunction.

Limit of Detection (LOD)

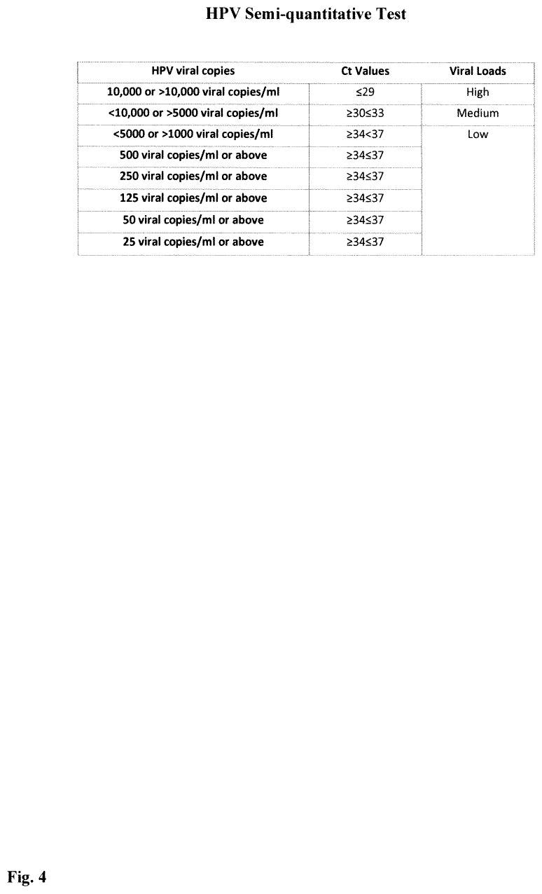

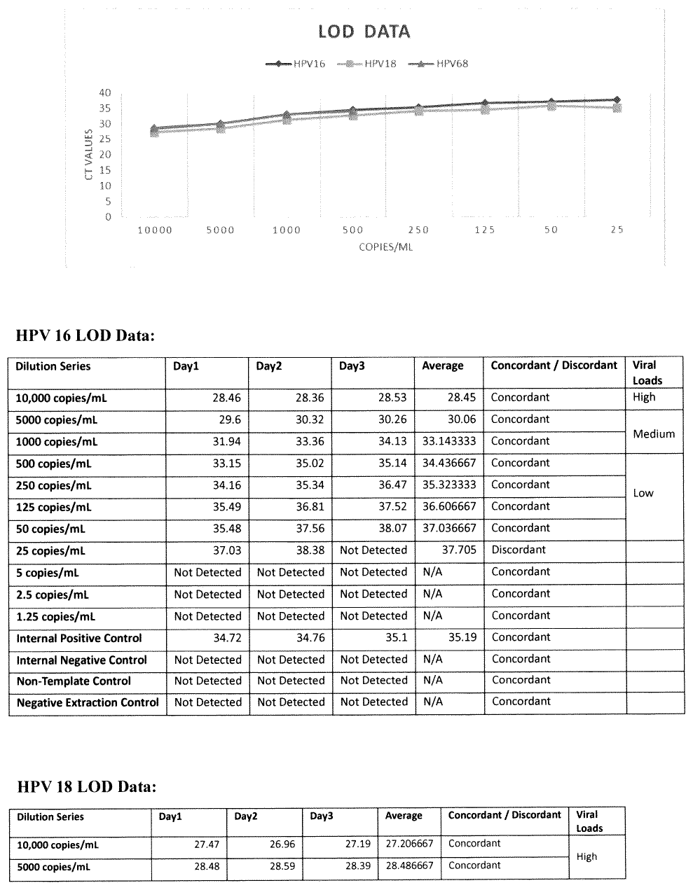

FIG. 4 presents LOD data for HPV 16, HPV 18 and HPV 68. This graph shows the LOD for HPV 16, HPV 18, and HPV 68 based on the corresponding Ct values and HPV copy numbers. The LOD represents the lowest viral copy number that can be reliably detected using the given testing method.

The detection of HPV nucleic acid molecules in a urine sample indicates the presence of an HPV infection in the patient. In a real time PCR assay a positive reaction is detected by accumulation of a fluorescent signal. The Ct (cycle threshold) is defined as the number of cycles required for the fluorescent signal to exceed background levels. Ct levels are inversely proportional to the amount of target nucleic acid in the sample (i.e., the lower the CT level the greater the amount of target nucleic acid in the sample).

LOD was performed as an analytical validation to provide data representing the range at which specific analytes can be detected based off the input being targeted. In other words, the Ct amplification value should reflect the amount of viral RNA being inputted into the assay. LOD for this analytical validation was evaluated using whole organism HPV 16, 18 and 68 (Exact Diagnostics LLC) at 1.25 copies/ml input in the Cobas® 6800/8800 Systems for HPV testing. The dilution series was 10,000 copies/ml, 5000 copies/mL, 1000 copies/mL, 500 copies/mL, 250 copies/mL 125 copies/mL, 50 copies/mL, 25 copies/mL, 5 copies/mL, 2.5 copies/mL, and 1.25 copies/mL. Samples were run three times, on three days, for accuracy reasons. Ct values for assay replicates were averaged, and the averages were plotted. The LOD was determined between 1.25-10,000 copies/mL, depending on the uniformity of Ct value of detectable copy number.

The stability and reproducibility of the assay using urine samples was analyzed with a total of 20 samples, including five HPV negative, and fifteen HPV spiked positive samples over a course of seven days. The samples were stored at 2-8° C. All 20 samples were run on Cobas 6800. The study was over a course of seven days at room temperature 20-22° C. (68-72° F.). The urine sample stability was measured based on the detection of internal control β globulin, and the urine sample was considered valid as long as the β globulin was detectable the sample according to protocol provided by manufacturer Roche Diagnostics. Stability is shown in FIG. 2. The Ct values were recorded and plotted for the complete duration of the stability experiment. For HPV 16 there was a shift of approximately 4 Ct values was observed, while for HPV 18 an HPV 68 a shift of 2 Ct values was observed. The reproducibility of the assay was assessed by calculating the coefficient of variation (CV %). The CV % was calculated between 4.2%-5.1% for HPV 16, 1.9%-3.6% for HPV 18, 2.3%-3.6% for HPV 68 and 1.2%-4.4% for β globulin which lies within the acceptable range. For β globulin this shift was 4 Ct values. The clinical sensitivity, specificity, overall percent agreement and accuracy were evaluated for all the samples and calculated as percentages. The Ct values were expressed as mean and standard deviation.

The method has good accuracy and excellent specificity for detection of HPV from urine in men and is an alternative to cell sample cytology testing. The method has utility for HPV self-sampling in men.

The method has a sensitivity of 90% or greater. The method has 100% specificity. The urine HPV detection method had a sensitivity of 90% for HPV 68, 93% for HPV 16 and 94% for HPV 18 and a specificity of 100% for HPV 68, HPV 16 and HPV 18 using Cobas® 6800. These sensitivities are much higher than those previously reported for urine PCR HPV detection. Some samples contained combined HPV 16 and 18 or other combined serotypes. The New Jersey state guidelines for validation were followed. The sensitivity and specificity of the assay is comparable with HPV detection using clinician-obtained cervical samples. The results are also consistent with sensitivity and specificity thresholds considered adequate for cervical screening.

The study subjects were both male and female. Cis and transgender men are the main carriers for HPV and transfer the infection to women and other men. HPV screening is equally important for men and non-binary people as for cisgender women.

The method uses florescence for nucleic acid labeling. In some embodiments, other methods of observing or quantifying signals indicating nucleic acid sequences can be used or combined with florescence, for example, radioactivity, colorimetry, gravimetry, X-ray diffraction or adsorption, magnetism, enzymatic activity and the like. The signal can be detected by methods appropriate to the type of signal.

The assay can be considered clinically validated for screening purposes for both vaccinated and unvaccinated male patients. The validation of this urine-based HPV DNA test is of immense value not only in screening large population and children but also for HPV vaccine monitoring in adolescents.

Reproducibility Criteria were Met.

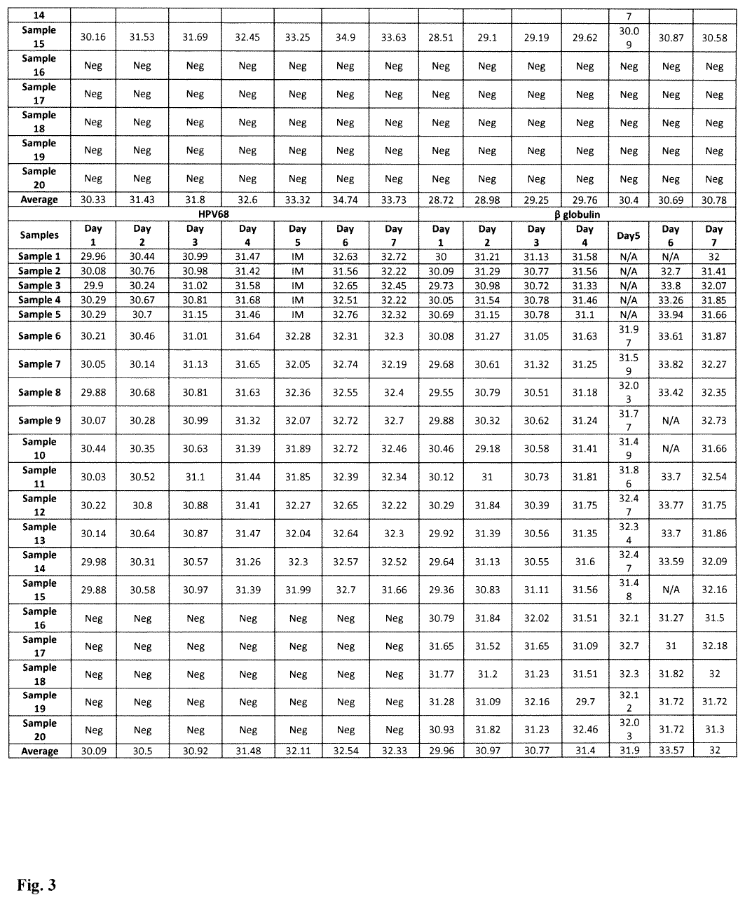

Semi-quantitative test for viral load is achieved based on the number of HPV nucleic acid copies. The software interpretation of Ct value in relation to control (know copy number) provides the viral load category as low medium or high. Ct value of 29 or less is high HPV load, Ct value of 30 to 33 is categorized as medium HPV load, and Ct value of 34 to 37 is low HPV load. The semi-quantitative test is shown in FIG. 3.

Valid test results were obtained for all the positive spiked and negative urine samples using Cobas 6800. The LOD for HPV 16 and HPV 18 was established at 50 copies/mL with three out of three positive calls at an average Ct value of 37.03±1.37 and 35.56±0.8, respectively. Similarly, the LOD for HPV 68 was measured to be 1000 copies/mL with three out of three positive cells at an average Ct cut-off value of 33.03±0.31. No HPV was detected for negative controls, and the overall concordance was found to be 100% for all the urine samples tested. The LOD results of HPV 16, HPV 18, and HPV 68 plotted against their mean Ct values are shown in FIG. 4.

Cobas 6800 detects both HPV16 and HPV18 at a minimum concentration of 50 copies/mL with an average Ct value of 37 and 35.6, respectively. Both the HPV genotypes produced two out of three positive calls at a concentration of 25 copies/mL which were considered discordant results. These genotypes were completely undetected at much lower copy numbers such as 5, 2.5 and 1.25 copies/mL. The LOD for HPV 68 was 1000 copies/mL at an average Ct value of 33. There were two out of three and one out of three positive calls recorded at lower copy numbers i.e. 500 and 250 copies/mL, respectively. The internal positive control was also run with all three genotypes and showed high concordance at an average Ct value of 35. No HPV was detected for any of the negative controls. The Cobas 6800 assay showed good concordance in urine samples consistent with HPV detection in clinician-obtained cervical samples.

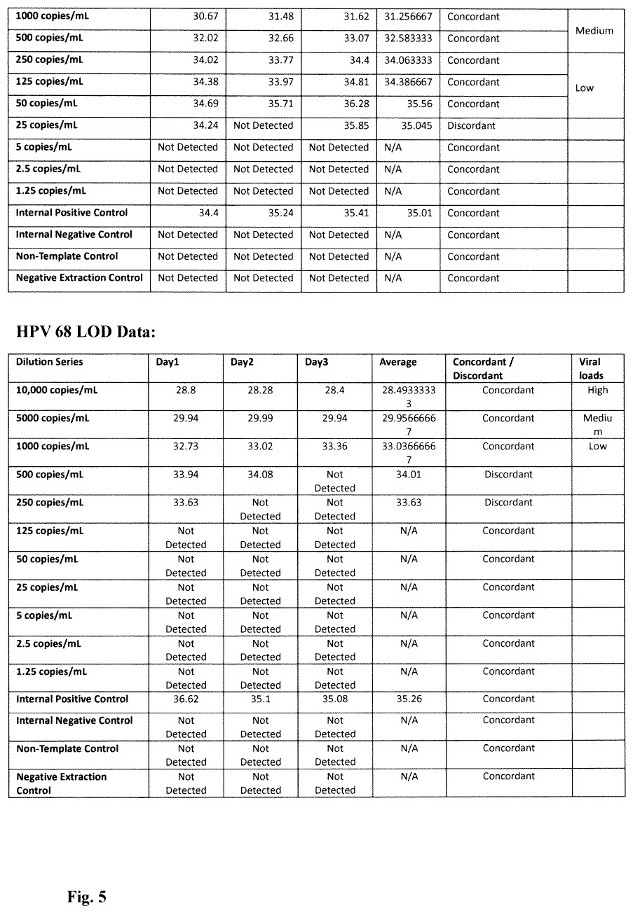

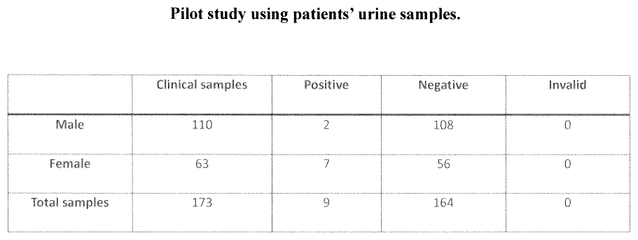

FIG. 5 is the pilot validation study on clinical urine samples for HPV detection. The table presents the results of a pilot study conducted to evaluate the sensitivity of an HPV detection assay using urine samples from patients with a known history of reproductive and urinary health issues. The study included a total of 173 samples including 110 male and 63 female urine samples. Out of the 110 male patients, 2 tested positive for HPV, while in the 63 female patients, 7 tested positive. The remaining samples in both male and female groups tested negative for HPV.

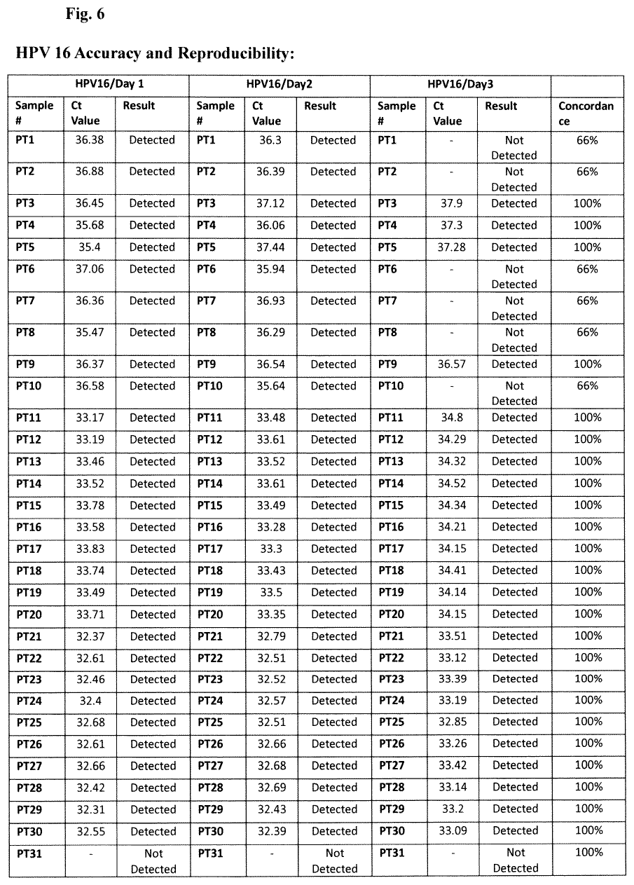

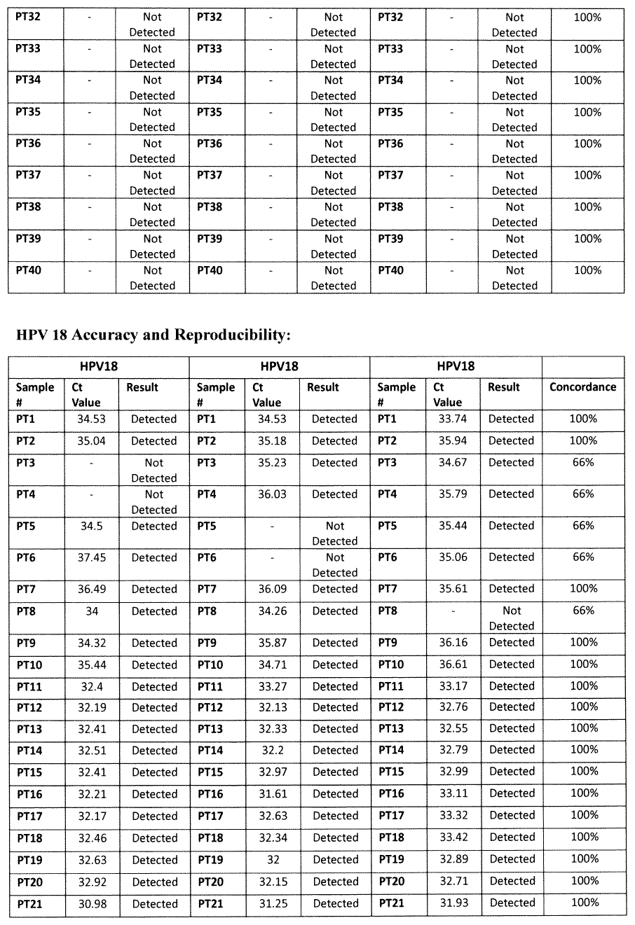

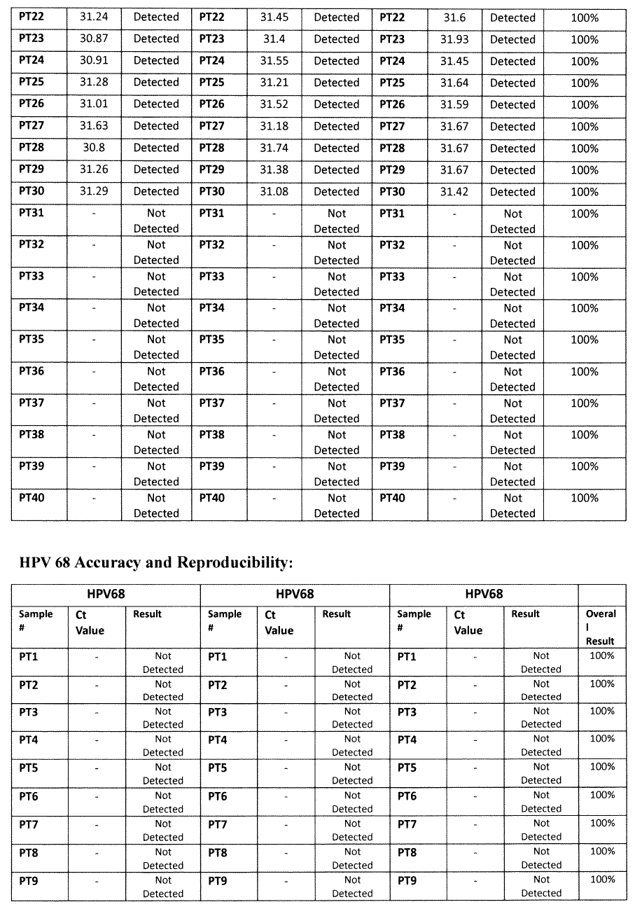

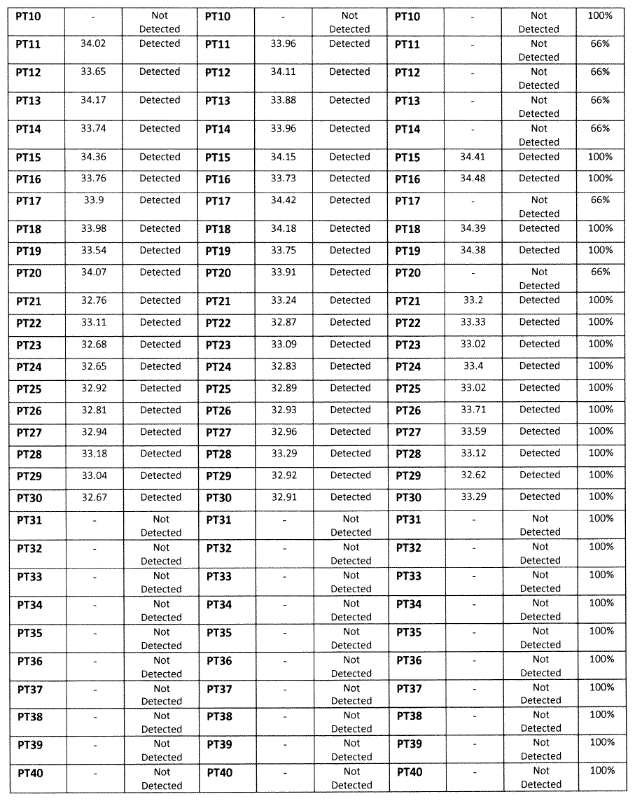

The table in FIG. 6 shows the data on accuracy and reproducibility of urine-based HPV detection using the Cobas. A total of 40 samples including 30 spiked positive samples and 10 negative controls were analyzed over a course of three days to detect HPV 16, HPV 18, and HPV 68. For all the three genotypes of HPV the table presents the Ct (Cycle threshold) values and corresponding results obtained on each day of analysis. The Ct value indicates the cycle number at which the fluorescence signal crosses a set threshold, providing a measure of the target DNA quantity in the sample. The results are categorized as “Detected” or “Not Detected” based on a predefined threshold. The table also includes a column for “Concordance,” which represents the percentage of samples where the results were consistent across all three days of analysis.

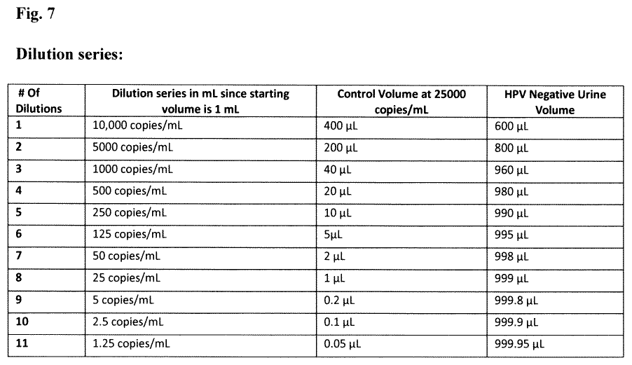

FIG. 7 presents the dilution series used to establish the limit of detection for HPV in urine samples. The dilution series was prepared with HPV positive control and HPV negative urine. The table presents the details of each dilution, including the number of dilutions, the corresponding dilution series in mL, the volume of the positive control at a concentration of 25,000 copies/mL, and the volume of HPV-negative urine used. Dilutions were prepared by decreasing the volume of the positive control and increasing the volume of HPV-negative urine, resulting in decreasing concentrations (copies/ml) of HPV. Each dilution maintained a final volume of 1 ml.

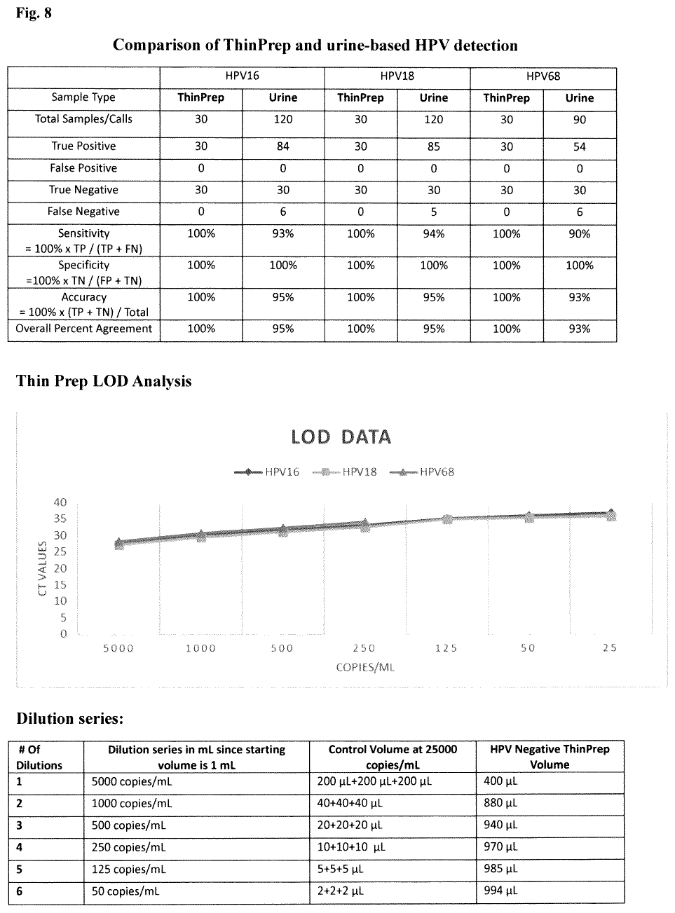

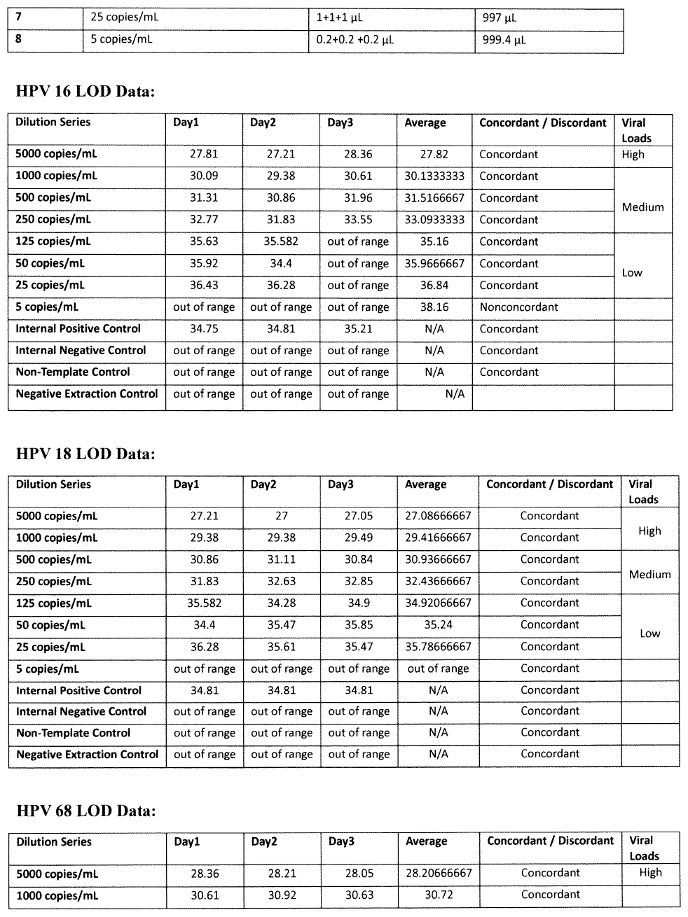

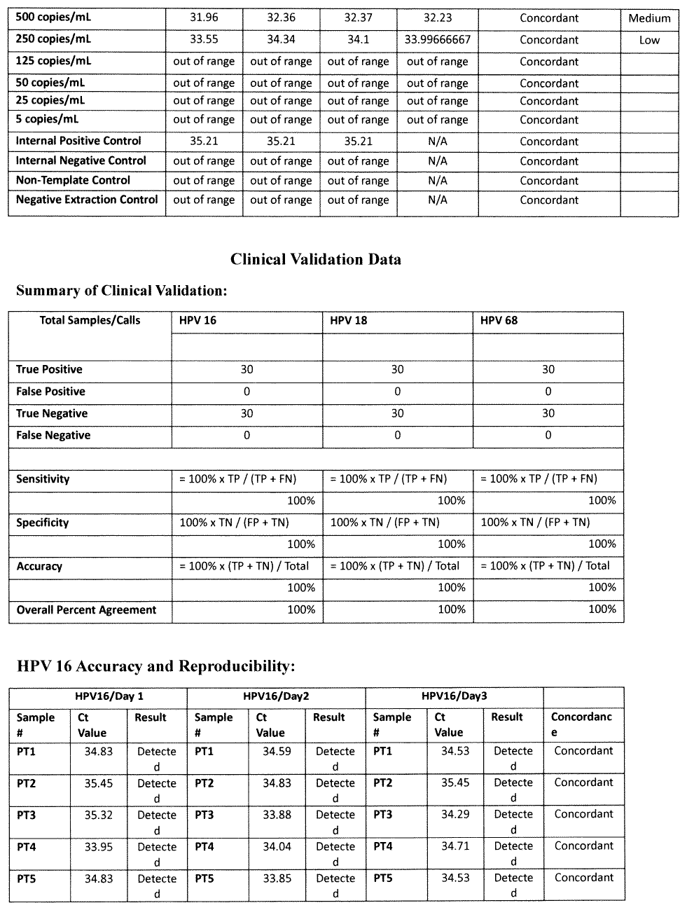

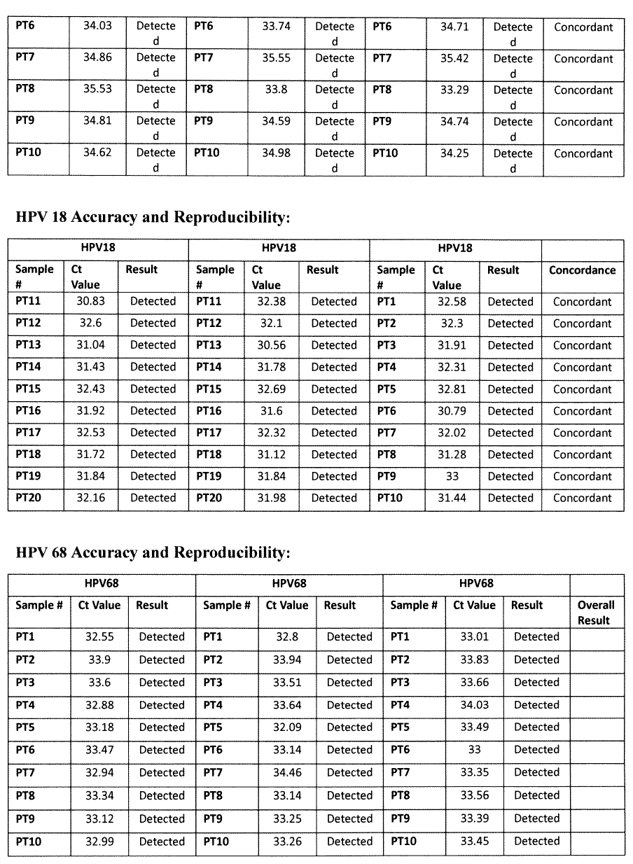

The table in FIG. 8 shows the comparison of diagnostic parameters for detecting HPV 16, 18, and 68 in urine and ThinPrep samples (classical/traditional method). It was found that the sensitivity of urine-based HPV detection using the Cobas 6800 for these HPV types ranged from 90% to 94%, whereas the sensitivity for ThinPrep was 100% for all three types. The calculated specificity for both sample types was found to be 100%. Additionally, the accuracy and overall percent agreement for urine was evaluated, which ranged from 93% to 95%, in comparison with 100% for ThinPrep. Based on the results, we can conclude that both methods, urine-based HPV detection using the Cobas 6800 and the ThinPrep collection method, exhibited comparable sensitivity, specificity, accuracy, and overall percent agreement. The data in FIG. 8 shows that in a comparison between the two different collection methods, the Thin Prep and urine analyte have the same sensitivity, specificity, accuracy and overall percent agreement across the board.

Amplification as in step three (3) is achieved in three (3) steps which consist of (1) denaturation, in which double-stranded DNA templates are heated to separate the strands; (2) annealing, in which short DNA molecules called primers bind to flanking regions of the target DNA; and (3) extension, in which DNA polymerase extends the 3′ end of each primer along the template strands. These steps are repeated (“cycled”) 25-35 times to exponentially produce exact copies of the target DNA. Once those cycles are completed, the amplified DNA is given a primer which illuminates certain DNA pairs in the sample. This then gives the machine a sense of how much DNA or RNA is present in the sample.

As used herein, the term “PCR” (polymerase chain reaction) describes a method for increasing the concentration of a segment of a target sequence in a mixture of DNA without cloning or purification. The process for amplifying the target sequence consists of introducing a large excess of two oligonucleotide primers to the DNA mixture containing the desired target sequence, followed by a precise sequence of thermal cycling in the presence of a DNA polymerase. The two primers are complementary to their respective strands of the double stranded target sequence. To effect amplification, the mixture is denatured and the primers then annealed to their complimentary sequences within the target molecule. Following annealing, the primers are extended with a polymerase so as to form a new pair of complimentary strands. These steps are repeated many times to obtain a high concentration of an amplified segment of the desired target sequence.

According to one embodiment the PCR technology may be real-time PCR (quantitative PCR or qPCR), reverse-transcriptase (RT-PCR), PCR-SSCP (single strand conformation polymorphism), ligase chain reaction (LCR), multiplex PCR or nested PCR.

Other types of amplification tests which may be used with the present invention include, but are not limited to: reverse transcription polymerase chain reaction (RT-PCR); reverse transcription loop-mediated isothermal amplification (RT-LAMP); and isothermal amplification including: nicking endonuclease amplification reaction (NEAR), transcription mediated amplification (TMA), loop-mediated isothermal amplification (LAMP), helicase-dependent amplification (HDA), clustered regularly interspaced short palindromic repeats (CRISPR), and strand displacement amplification (SDA).

According to one aspect of the invention, the PCR amplification process may use, but not be limited to, Thermal Cycle Machines. Specific examples of thermal cycle machine which may be used include those made by Roche Diagnostics (6800, 8800 COBAS), Vela Diagnostics (Senotas), Thermo Fischer (Quant Studio Flex 12 K).

Any PCR test may be used including any Thin Prep tests. Examples of such tests include, but are not limited to, Abbott Real Time, Cervista HPV HR (Hologic GEN-PROBE), and Digene HC2 (Hybrid Capture 2) (Qlagen).

The detection of HPV can be of a single type, e.g. HPV 18 or may be of two or more types, such as HPV 16, 18, or 68, or HPV 16, 18, 31, 33, 35, 39, 45, 51, 52, 56, 58, 59, and 68. In some cases, the detection is of high-risk types. Non-limiting examples of such high-risk types are HPV 16, 18 and 68.

In some embodiments, the detection of HPV infection is in male patients. In other embodiments, the detection is in female or transgender patients as several of the urine samples were from female patients. In yet other embodiments, the detection is in children or adolescents.

The present disclosure will be described with respect to particular embodiments and with reference to certain drawings, but the present disclosure is not limited thereto but only by the claims. The drawings described are only schematic and are non-limiting.

Reference throughout this specification to “one embodiment” or “an embodiment” means that a particular feature, structure or characteristic described in connection with the embodiment is included in at least one embodiment of the present disclosure. Thus, appearances of the phrases “in some embodiments,” “in one embodiment,” or “in an embodiment” in various places throughout this specification are not necessarily all referring to the same embodiment, but may. Furthermore, the particular features, structures, or characteristics may be combined in any suitable manner, as would be apparent to one of ordinary skill in the art from this disclosure, in one or more embodiments.

The terminology used herein is for the purpose of describing particular aspects only and is not intended to be limiting of the disclosure. As used herein, the singular forms “a,” “an” and “the” are intended to include the plural forms as well, unless the context clearly indicates otherwise. It will be further understood that the terms “comprises” and/or “comprising,” when used in this specification, specify the presence of stated features, integers, steps, operations, elements, and/or components, but do not preclude the presence or addition of one or more other features, integers, steps, operations, elements, components, and/or groups thereof.

The description of the present disclosure has been presented for purposes of illustration and description, but is not intended to be exhaustive or limited to the disclosure in the form disclosed. Many modifications and variations will be apparent to those of ordinary skill in the art without departing from the scope and spirit of the disclosure. The aspects of the disclosure herein were chosen and described in order to best explain the principles of the disclosure and the practical application, and to enable others of ordinary skill in the art to understand the disclosure with various modifications as are suited to the particular use contemplated.

Although the subject matter has been described in language specific to structural features and/or methodological acts, it is to be understood that the subject matter defined in the appended claims is not necessarily limited to the specific features or acts described above. Rather, the specific features and acts described above are disclosed as example forms of implementing the claims.

The disclosure presented herein is believed to encompass at least one distinct invention with independent utility. While the at least one invention had been disclosed in exemplary forms, the specific embodiments thereof as described and illustrated herein are not to be considered in a limiting sense, as numerous variation are possible. Equivalent changes, compositions and methods may be made within the scope of the present disclosure, achieving substantially similar results. The subject matter of the at least one invention includes all novel and on-obvious combinations and sub-combinations of the various elements, features, functions and/or properties disclosed herein and their equivalents.

What is claimed is:

1. A gender-neutral polymerase chain-reaction (PCR) molecular amplification method for detecting high-risk HPV genotypes in urine analyte in a patient, the method comprising:
providing a unaltered urine specimen from a male, non-binary or transgender patient;
extracting DNA from a cell;
using a PCR amplification process;
testing the DNA by Cobas® HPV test using Cobas® 6800 or 8800 assay; and
determining a positive or negative result for HPV detection.

2. The method of claim 1, wherein the high-risk HPV genotypes is selected from the group consisting of HPV 16, HPV 18 and HPV 68.

3. The method of claim 2, wherein the urine HPV detection method has a sensitivity ranging from 90% to 94% and a specificity of 100%.

4. The method of claim 3, wherein the urine HPV detection method has a sensitivity of 90% for HPV 68, 93% for HPV 16 and 94% for HPV 18.

5. The method of claim 1, having good concordance in urine samples consistent with HPV detection in clinician-obtained cervical samples.

6. The method of claim 5, wherein the urine HPV detection method has an accuracy and overall percent agreement ranging from 93% to 95% in comparison with 100% for a ThinPrep cervical test.

7. The method of claim 2, wherein said detecting of HPV 16 and HPV 18 is at a minimum concentration of 50 copies/mL with an average Ct value of 37 and 35.6.

8. The method of claim 2, wherein said detecting of HPV 68 is at a minimum concentration of 1000 copies/mL at an average Ct value of 33.

9. The method of claim 1, wherein the polymerase chain-reaction (PCR) molecular amplification method is selected form the group consisting of real-time PCR (quantitative PCR or qPCR), reverse-transcriptase (RT-PCR), PCR-SSCP (single strand conformation polymorphism), ligase chain reaction (LCR), multiplex PCR and nested PCR.

10. The method of claim 1, wherein the polymerase chain-reaction (PCR) molecular amplification method is real-time PCR using Cobas 6800.

11. The method of claim 1, wherein the patient is selected from the group consisting of a non-binary woman, a non-binary male, a transgender male, a transgender woman, a child, and an adolescent.

12. A home-based polymerase chain-reaction (PCR) molecular amplification method for detecting high-risk HPV genotypes in urine analyte in a male patient, the method comprising:
allowing a male patient to self-sample and return a urine sample to a laboratory;
extracting DNA from a cell;
using a PCR amplification process;
testing the DNA by Cobas® HPV test using Cobas® 6800 or 8800 assay; and
determining a positive or negative result for HPV detection.

13. The method of claim 12, further comprising a coefficient of variation (CV %) of between 4.2% to 5.1% for HPV 16, 1.9% to 3.6% for HPV 18, and 2.3% to 3.6% for HPV 68.

14. The method of claim 12, wherein the PCR molecular amplification method is integrated with data management systems to allow for epidemiological surveillance and control of disease outbreaks.

15. The clinically validated assay of claim 12, further comprising a kit.

16. A clinically validated PCR assay for high-risk genotype HPV in a urine analyte in male, non-binary and transgender patient, said assay comprising:
nucleic acid extraction and release from the urine analyte;
binding the nucleic acid to a silica surface of added magnetic glass particles to purify it;
amplifying the nucleic acid by PCR reaction using thermostable DNA polymerase; and
assigning the results as positive, negative or invalid with an automated management software.

17. The clinically validated PCR assay of claim 16, which correlates with clinical significance.

18. The clinically validated PCR assay of claim 16, wherein the high-risk HPV genotypes is selected from the group consisting of HPV 16, HPV 18 and HPV 68.

19. The clinically validated PCR assay of claim 16, having 100% specificity and a sensitivity which ranges from 90% to 94%.

20. The clinically validated assay of claim 16, wherein the assay is useful for mass screening and self-sampling of a specimen via a diagnostic kit.
